(19) United States
(12) Patent Application Publication
(10) Pub. No.: US 2012/0095002 A1
(43) Pub. Date: Apr. 19, 2012

(54) ISOXAZOLE-5-CARBOXAMIDE DERIVATIVES

(75) Inventors: Paul David Ratcliffe, Lanarkshire (GB); Ronald Palin, Banton (GB)

(73) Assignee: N.V. Organon, Oss (NL)

(21) Appl. No.: 13/256,561

(22) PCT Filed: Feb. 2, 2010
(86) PCT No.: PCT/EP10/51251

§ 371 Date: Dec. 2, 2011

Related U.S. Application Data
(60) Provisional application No. 61/149,721, filed on Feb. 4, 2009.

(30) Foreign Application Priority Data
Feb. 4, 2009 (EP) 09152061.9

(51) Int. Cl. A61K 31/5377; C07D 413/12; C07D 413/14; A61P 13/02; A61K 31/422; A61P 29/00; A61P 11/00; C07D 261/18; A61K 31/42

(52) U.S. Cl. 514/236.8; 548/248; 544/137; 514/378

(57) ABSTRACT

The present invention relates to isoxazole-5-carboxamide derivative having the general Formula (I), or a pharmaceutically acceptable salt thereof, to pharmaceutical compositions comprising the same, as well as to the use of said isoxazole-5-carboxamide derivatives for the treatment of TRPV1 mediated disorders, such as acute and chronic pain disorders, acute and chronic neuropathic pain, acute and chronic inflammatory pain, respiratory diseases, and lower urinary tract disorders.

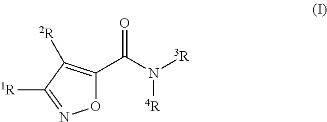

The present invention relates to isoxazole-5-carboxamide derivatives, to pharmaceutical compositions comprising the same and to the use of these isoxazole-5-carboxamide derivatives in the treatment of TRPV1 related disorders.

The vanilloid receptor (VR1 or TRPV1), a non-selective ligand-gated cation channel belonging to the Transient Receptor Channel family (TRP family) of cation channels, is highly expressed on the peripheral termini of small diameter sensory neurones innervating many tissues including skin, bladder, airway and gastrointestinal tract. More specifically TRPV1 receptors are located on a subset Aδ and C fibres, the afferents commonly associated with nociception (Mezey et al., Proc. Natl. Acad. Sci. 97, 3655-3660, 2000). Characterisation of this channel at the molecular level identified it as the target of the vanilloid capsaicin, the main pungent constituent of hot chilli peppers (Caterina et al., Nature 389, 816-824, 1997). Indeed, sensitivity to capsaicin has been used for many years as a marker of nociceptor activity. These, polymodal nociceptors are activated by multiple noxious stimuli including chemical, mechanical and thermal. Study of the functional properties of TRPV1 demonstrated that this receptor shares many properties common to nociceptors including activation by thermal stimuli (>43° C.) and chemicals (including capsaicin and endovanilloids such as N-arachidonoyl-dopamine (NADA) and lipoxygenase metabolites), as well as sensitisation and activation by acidification. Furthermore, inflammatory mediators (including ATP and bradykinin) have been shown to functionally sensitise TRPV1 in vitro. This evidence suggests that TRPV1 has an integral role in the polymodal detection of noxious stimuli and contributes to the transduction of inflammatory pain responses and potentially also peripheral tissue injury (reviewed in Di Marzo et al., Curr. Opin. Neurobiol. 12, 372-379, 2002).

A role for TRPV1 in the detection of painful stimuli is also inferred from data in gene knockout mice. Mice null for TRPV1 show attenuated development of behavioural thermal hyperalgesia after an inflammatory insult (Caterina et al., Science 288, 306-313, 2000, Davis et al., Nature 405, 183-187, 2000). Small diameter sensory neurones from these animals also show altered responses to thermal and acid stimuli. Moreover, altered expression and/or functional activity of TRPV1 has been demonstrated following inflammation and nerve injury in animals models (Amaya et al., Brian Res. 963, 190-196, 2003, Rashid et al., J. Pharm. Exp. Ther. 304, 940-948, 2003, Hong & Wiley, J. Biol. Chem. 280, 618-627, 2005).

In addition, to a role in pain transduction there is also growing evidence for a role for TRPV1 in regulating afferent and efferent function of sensory nerves and the function of non-neuronal cells. Indeed, altered bladder function, with a higher frequency of low amplitude, non-voiding bladder contractions and an increase in bladder capacity has been observed by in TRPV1 KO mice (Birder et al., Nat. Neurosci. 5, 856-860, 2002). This may involve neuronal TRPV1 and TRPV1 expressed on uroepithelial cells. Thus, there is clear evidence to suggest that agents modulating TRPV1 activity will have utility in not only in pain states and other diseases involving inflammation but also in conditions involving hyperactivity of primary sensory fibres (e.g. bladder overactivity and urge incontinence).

Isoxazole-3-carboxamide derivatives have been disclosed in the International Patent Application WO 2007/067710 (Amphora Discovery Corporation) as modulators of the TRPV1 receptor and useful in the treatment of TRPV1 mediated disorders, such as in the treatment of acute and chronic pain disorders, acute and chronic neuropathic pain, acute and chronic inflammatory pain, respiratory diseases, and lower urinary tract disorders. There remains a need for additional, more potent, compounds that are useful in the treatment of TRPV1 mediated disorders.

To this end the present invention provides isoxazole-5-carboxamide derivatives having the general Formula I

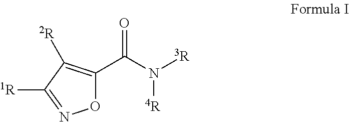

wherein

R₁ is phenyl or pyridyl, each of which optionally substituted by 1-3 substituents selected from halogen, (C₁₋₄)alkyl, halo(C₁₋₄)alkyl, (C₁₋₄)alkyloxy and halo(C₁₋₄)alkyloxy;

R₂ is halogen, (C₁₋₃)alkyl, hydroxy(C₁₋₃)alkyl, (C₁₋₄)alkyloxy(C₁₋₃)alkyl, (C₃₋₈)cycloalkyl, hydroxy(C₃₋₈)cycloalkyl or R₅R₆N(C₁₋₃)alkyl;

R₃ is (C₁₋₈)alkyl, halo(C₁₋₈)alkyl, hydroxy(C₁₋₈)alkyl, (C₂₋₈)alkenyl, (C₂₋₈)alkynyl, (C₃₋₁₀)-cycloalkyl, (C₃₋₈)cycloalkenyl or (C₃₋₈)cycloalkyl(C₁₋₃)alkyl, each cycloalkyl group optionally substituted by oxo, hydroxyimino, hydroxy, carboxy, cyano, (C₁₋₃)alkyl or hydroxy(C₁₋₃)alkyl; or

R₃ is a saturated 4-8-membered heterocyclic ring containing a heteroatom selected from O, S and SO₂, optionally substituted by hydroxyl or oxo;

R₄ is H or (C₁₋₄)alkyl; or

R₄ together with R₃ and the N to which they are bonded form a saturated 4-8 membered ring, optionally containing a further heteroatom selected from O, S and SO₂, the ring being optionally substituted by oxo, hydroxyimino, hydroxy, carboxy, carboxamido, (C₁₋₃)alkyl, hydroxy(C₁₋₃)alkyl or (C₁₋₃)-alkyloxy;

R₅ and R₆ are independently H, (C₁₋₆)alkyl, (C₃₋₆)cycloalkyl or (C₃₋₆)cycloalkyl-(C₁₋₃)alkyl, each alkyl group being optionally substituted with halogen, hydroxy or (C₁₋₄)alkyloxy; or

R₅ and R₆ form together with the nitrogen to which they are bonded a 5- or 6-membered saturated heterocyclic ring, optionally comprising a further heteroatom selected from O, S and SO₂; or a pharmaceutically acceptable salt thereof.

The term (C₁₋₃)alkyl used in the definition of Formula I means a branched or unbranched alkyl group having 1-3 carbon atoms, like propyl, isopropyl, ethyl and methyl.

The term hydroxy(C₁₋₃)alkyl means a branched or unbranched alkyl group having 1-3 carbon atoms substituted by 1 or 2 hydroxy groups, such as 3-hydroxypropyl, 2,3-dihydroxypropyl, 2-hydroxyethyl or hydroxymethyl.

The term (C₁₋₄)alkyl as used in the definition of Formula I means a branched or unbranched alkyl group having 1-4 carbon atoms, like butyl, isobutyl, tertiary butyl, propyl, isopropyl, ethyl and methyl.

The term halo(C₁₋₄)alkyl means a branched or unbranched alkyl group having 1-4 carbon atoms substituted by 1-3 halogens. A preferred halo(C₁₋₄)alkyl is CF₃.

In the term (C₁₋₄)alkyloxy, (C₁₋₄)alkyl has the meaning as defined above.

In the term halo(C₁₋₄)alkyloxy, halo(C₁₋₄)alkyl has the meaning as defined above.

The term (C₁₋₈)alkyl as used in the definition of Formula I means a branched or unbranched alkyl group having 1-8 carbon atoms, like octyl, hexyl, hexyl, pentyl, isopentyl, butyl, isobutyl, tertiary butyl, propyl, isopropyl, ethyl and methyl.

The term (C₂₋₈)alkenyl means a branched or unbranched alkenyl group having 2-8 carbon atomes, such as ethenyl, propen-2-yl, 2-methyl-propenyl, penten-4-yl and the like.

The term (C₂₋₈)alkynyl means a branched or unbranched alkynyl group having 2-8 carbon atomes, such as ethynyl, propyn-2-yl, pentyn-4-yl and the like.

The term (C₃₋₁₀)cycloalkyl means a cycloalkyl group having 3-10 carbon atoms, like cycloheptyl, cyclohexyl, cyclopentyl, cyclobutyl and cyclopropyl. Also included in this term are bicyclic cycloalkyl groups such as bicyclo[2,2,1]heptan-2-yl, bicyclo[2,2,1]hept-2-enyl, bicyclo[2,2,2]oct-5-enyl, and tricyclic alkyl groups such as adamantyl and the like.

The term (C₃₋₈)cycloalkenyl means a cycloalkenyl group having 3-8 carbon atoms, like cyclooct-3-yl, cyclohex-3-yl and cyclopent-2-yl.

The term a saturated 4-8-membered heterocyclic ring containing a further heteroatom selected from O, S and SO₂, as used in the definition of R₄ together with R₃ and the N to which they are bonded is exemplified by N-morpholinyl, N-thiomorpholinyl and N-thiazolidinyl.

The term a saturated 4-8-membered heterocyclic ring containing a heteroatom selected from O, S and SO₂, as used in the definition of R₃ of formula I is exemplified by tetrahydropyranyl, tetrahydrofuranyl, tetrahydrothiopyranyl, tetrahydrothienyl and N-morpholinyl.

The term halogen means F, Cl, Br or I. Preferred are F and Cl.

In one embodiment the invention provides isoxazole-5-carboxamide derivatives according to formula I, wherein

R₁ is phenyl, optionally substituted by 1-3 substituents selected from halogen, (C₁₋₄)alkyl, halo(C₁₋₄)alkyl, (C₁₋₄)alkyloxy and halo(C₁₋₄)alkyloxy;

R₂ is halogen, hydroxy(C₁₋₃)alkyl or R₅R₆N(C₁₋₃)alkyl;

R₃ is (C₁₋₈)alkyl, halo(C₁₋₈)alkyl, hydroxy(C₁₋₈)alkyl or (C₃₋₁₀)cycloalkyl, optionally substituted by hydroxy; or

R₃ is a saturated 4-8-membered heterocyclic ring containing a heteroatom selected from O, S and SO₂, optionally substituted by hydroxyl or oxo;

R₄ is H or (C₁₋₄)alkyl;

R₅ and R₆ are independently H, (C₁₋₆)alkyl, (C₃₋₆)cycloalkyl or (C₃₋₆)cycloalkyl-(C₁₋₃)alkyl, each alkyl group being optionally substituted with halogen, hydroxy or (C₁₋₄)alkyloxy; or

R₅ and R₆ form together with the nitrogen to which they are bonded a 5- or 6-membered saturated heterocyclic ring, optionally comprising a further heteroatom selected from O, S and SO₂.

In another embodiment the invention provides isoxazole-5-carboxamide derivatives of formula I wherein

R₁ is phenyl, substituted by 1 or 2 substituents selected from F, C₁ and CF₃;

R₂ is Cl, Br, hydroxy(C₁₋₃)alkyl or R₅R₆N(C₁₋₃)alkyl;

R₃ is (C₁₋₈)alkyl, halo(C₁₋₈)alkyl, hydroxy(C₁₋₈)alkyl or (C₃₋₁₀)cycloalkyl, optionally substituted by hydroxy; or

R₃ is a saturated 4-8-membered heterocyclic ring containing a heteroatom selected from O, S and SO₂;

R₄ is H or (C₁₋₄)alkyl;

R₅ and R₆ are independently H, (C₁₋₆)alkyl, (C₃₋₆)cycloalkyl or (C₃₋₆)cycloalkyl-(C₁₋₃)alkyl, each alkyl group being optionally substituted with halogen, hydroxy or (C₁₋₄)alkyloxy; or

R₅ and R₆ form together with the nitrogen to which they are bonded a 5- or 6-membered saturated heterocyclic ring, optionally comprising a further heteroatom selected from O, S and SO₂.

Specifically preferred isoxazole-5-carboxamide derivatives of the invention are:
4-chloro-N-((1R,3S)-3-hydroxycyclohexyl)-3-(4-(trifluoromethyl)phenyl)isoxazole-5-carboxamide;
4-bromo-N-cyclopentyl-3-(4-(trifluoromethyl)phenyl)isoxazole-5-carboxamide;
4-bromo-N-(tetrahydro-2H-pyran-4-yl)-3-(4-(trifluoromethyl)phenyl)isoxazole-5-carboxamide;
4-chloro-N-cyclopentyl-3-(4-(trifluoromethyl)phenyl)isoxazole-5-carboxamide;
4-chloro-3-(4-fluorophenyl)-N-((1R,3S)-3-hydroxycyclohexyl)isoxazole-5-carboxamide;
4-chloro-N-(tetrahydro-2H-pyran-4-yl)-3-(4-(trifluoromethyl)phenyl)isoxazole-5-carboxamide;
4-chloro-N-cyclopentyl-N-methyl-3-(4-(trifluoromethyl)phenyl)isoxazole-5-carboxamide;
(S)-4-chloro-N-(3-methylbutan-2-yl)-3-(4-(trifluoromethyl)phenyl)isoxazole-5-carboxamide;
(R)-4-chloro-N-(1-hydroxybutan-2-yl)-3-(4-(trifluoromethyl)phenyl)isoxazole-5-carboxamide;
(S)-4-bromo-3-(4-(trifluoromethyl)phenyl)-N-(1,1,1-trifluoropropan-2-yl)isoxazole-5-carboxamide;
(S)-4-chloro-3-(4-(trifluoromethyl)phenyl)-N-(1,1,1-trifluoropropan-2-yl)isoxazole-5-carboxamide;
4-chloro-N-(3,3-difluorocyclobutyl)-3-(4-(trifluoromethyl)phenyl)isoxazole-5-carboxamide;
(S)-4-chloro-3-(4-fluorophenyl)-N-(1,1,1-trifluoropropan-2-yl)isoxazole-5-carboxamide;
4-chloro-3-(4-chloro-3-fluorophenyl)-N-(cis)-2-hydroxycyclohexyl)isoxazole-5-carboxamide;
4-chloro-3-(4-chloro-3-fluorophenyl)-N-((1R,3S)-3-hydroxycyclohexyl)isoxazole-5-carboxamide;
N-cyclopentyl-3-(3-fluoro-4-(trifluoromethyl)phenyl)-4-(hydroxymethyl)isoxazole-5-carboxamide;
4-chloro-3-(3-fluoro-4-(trifluoromethyl)phenyl)-N-(trans)-2-hydroxycyclohexyl)-isoxazole-5-carboxamide;
4-chloro-3-(3-fluoro-4-(trifluoromethyl)phenyl)-N-(tetrahydro-2H-pyran-4-yl)-isoxazole-5-carboxamide;
N-cyclopentyl-4-((ethyl(isopropyl)amino)methyl)-3-(3-fluoro-4-(trifluoromethyl)-phenyl)isoxazole-5-carboxamide; and
3-(3-fluoro-4-(trifluoromethyl)phenyl)-4-(morpholinomethyl)-N-(tetrahydro-2H-pyran-4-yl)isoxazole-5-carboxamide; or a pharmaceutically acceptable salt thereof.

The isoxazole-5-carboxamide derivatives of the invention may be prepared by methods known in the art of organic chemistry in general.

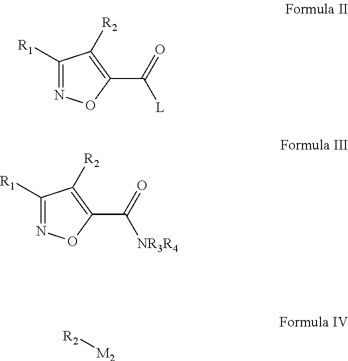

Isoxazole-5-carboxamide derivatives of Formula I may for instance be prepared from compounds of Formula II wherein L is a leaving group, such as a halogen or an acyloxy group, and wherein R₁ and R₂ have the meaning as previously defined, by nucleophilic displacement of the leaving group with an amine of formula NHR₃R₄. Compounds of Formula II where L is an acyloxy group may be prepared from compounds of Formula II where L is hydroxy, by reaction with for example chloroformate in the presence of a base such as N-methylmorpholine.

Isoxazole-5-carboxamide derivatives of Formula I may be prepared from compounds of Formula II wherein L is hydroxy, by reaction with, for example, oxalyl chloride with or without the presence of a catalyst such as N,N-dimethylformamide and further treatment with the appropriate amine NHR₃R₄ (J. Am. Chem. Soc., Vol. 108, No. 22, 6950-6960, 1986).

Isoxazole-5-carboxamide derivatives of Formula I may be prepared from compounds of Formula II where L is hydroxy, by treatment with one or more standard (peptide) coupling reagents well known in the art, such as O-(7-azabenzotriazol-1-yl)-N,N,N′,N′-tetramethyluronium hexafluorophosphate (HATU), dicyclohexylcarbodiimide (DCC), diisopropylcarbodiimide (DIC), or (benzotriazol-1-yl-oxy-trispyrrolidinophosphonium-hexafluorophosphate (PYBOP) and further treatment with the appropriate amine NHR₃R₄ (J. Am. Chem. Soc., Vol. 108, No. 22, 6950-6960, 1986).

Isoxazole-5-carboxamide derivatives of Formula I may be prepared from compounds of Formula II where L is acyloxy, by treatment with the appropriate amine NHR₃R₄, in an appropriate solvent, at temperatures between 50 to 200° C. using either conventional or microwave heating and a reaction time between 5 minutes and 30 hours.

In the alternative, compounds of Formula I may be prepared from compounds of Formula III where X is halogen by treatment with compounds of Formula IV, wherein R₂ is as previously defined and wherein M₂ is a boronic acid or a boronic acid ester, using a Suzuki reaction (Chem. Rev. 95, 2457-2483, 1995) or a modification thereof.

Compounds of Formula IV which serve as starting materials are commercially available or may be prepared by a variety of methods known in the art.

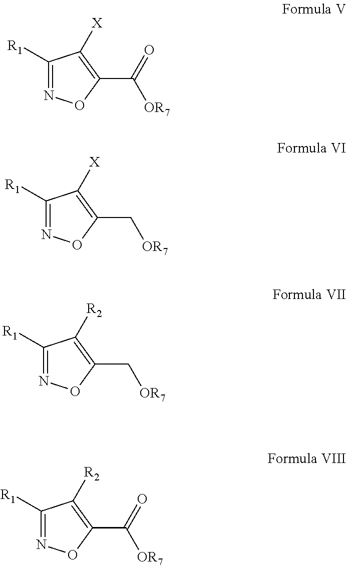

Compounds of Formula II, where L is alkoxy, may be prepared from compounds of Formula V wherein R₁ has the previously given meaning and R₇ is H or (C₁₋₆)alkyl and wherein X is halogen, by treatment with compounds of Formula IV, where M₂ is a boronic acid or a boronic acid ester, using a Suzuki reaction (Chem. Rev. 95, 2457-2483, 1995) or a modification thereof.

Compounds of Formula VI, where X is halogen may be prepared from compounds of Formula VII, using methods well known in the art for halogenating heterocyclic rings. Such as methods described in the general reference Davies, D. T. Aromatic Heterocyclic Chemistry (Oxford University Press: Oxford 1995).

It is well known in the art that compounds of Formula VII, where R₇ has the previously given meaning, can be prepared from compounds of Formula VIII, by reduction using suitable reducing agents, as described in Burke D. S., Danheiser, R. L. Handbook of Reagents for Organic Synthesis: Oxidising and Reducing agents (Wiley: New York, 1999).

Furthermore, compounds of Formula VII where R₇ has the previously given meaning, may be prepared by reaction of compounds of Formula IX, wherein R₈ is CH₂R₇ and R₂ has the previously given meaning or can be a carboxylic acid ester, in the presence of compounds of Formula X in a suitable solvent as described in the general reference Davies, D. T. Aromatic Heterocyclic Chemistry (Oxford University Press: Oxford 1995). Furthermore, compounds of Formula VIII where R₇ has the previously given meaning, may be prepared by reaction of compounds of Formula IX, wherein R₈ is CO₂R₇ and R₂ has the previously given meaning or can be a carboxylic acid ester, in the presence of compounds of Formula X in a suitable solvent as described in the general reference Davies, D. T. Aromatic Heterocyclic Chemistry (Oxford University Press: Oxford 1995).

Compounds of Formula IX which serve as starting materials are commercially available or may be prepared by a variety of methods known in the art.

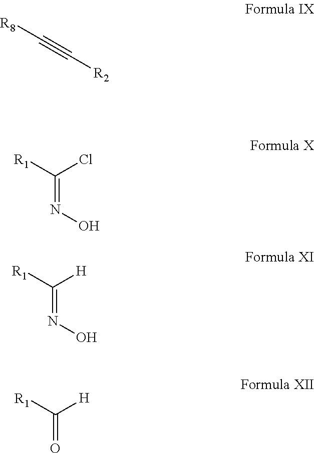

Compounds of Formula X may be prepared from compounds of Formula XI by treatment with but not restricted to, for example, N-chlorosuccinimide.

Compounds of Formula XI, where R₁ has the previously given meaning, may be prepared from compounds from compounds of Formula XII, by treatment with hydroxylamine in a suitable solvent.

The skilled person will likewise appreciate that various isoxazole-5-carboxamide derivatives of Formula I may be obtained by appropriate conversion reactions of functional groups corresponding to certain of the substituents R₃-R₄. For example, compounds of Formula I wherein R₃ or R₄ is an optionally substituted alkyl or cycloalkyl group, may be prepared by the reaction of a compound of Formula I wherein R₃ or R₄ is hydrogen with an appropriately functionalised alkyl or cycloalkyl halide, in the presence of a base such as potassium carbonate.

The isoxazole-5-carboxamide derivatives of Formula I and their salts may contain at least one centre of chirality, and exist therefore as stereoisomers, including enantiomers and diastereomers. The present invention includes the aforementioned stereoisomers within its scope and each of the individual R and S enantiomers of the compounds of Formula I and their salts, substantially free, i.e. associated with less than 5%, preferably less than 2%, in particular less than 1% of the other enantiomer, and mixtures of such enantiomers in any proportions including the racemic mixtures containing substantially equal amounts of the two enantiomers.

Methods for asymmetric synthesis or chiral separation whereby the pure stereoisomers are obtained are well known in the art, e.g. synthesis with chiral induction or starting from commercially available chiral substrates, or separation of stereoisomers, for example using chromatography on chiral media or by crystallisation with a chiral counter-ion.

The present invention also embraces isotopically-labelled isoxazole-5-carboxamide derivatives of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Pharmaceutically acceptable salts may be obtained by treating a free base of a compound of Formula I with a mineral acid such as hydrochloric acid, hydrobromic acid, phosphoric acid and sulfuric acid, or an organic acid such as for example ascorbic acid, citric acid, tartaric acid, lactic acid, maleic acid, malonic acid, fumaric acid, glycolic acid, succinic acid, propionic acid, acetic acid and methane sulfonic acid.

The compounds of the invention may exist in unsolvated as well as in solvated forms with pharmaceutically acceptable solvents such as water, ethanol and the like. In general, the solvated forms are considered equivalent to the unsolvated forms for the purpose of the invention.

The present invention further provides pharmaceutical compositions comprising an isoxazole-5-carboxamide derivative of the invention, or a pharmaceutically acceptable salt thereof, in admixture with pharmaceutically acceptable auxiliaries, and optionally other therapeutic agents. The term “acceptable” means being compatible with the other ingredients of the composition and not deleterious to the recipients thereof. Compositions include e.g. those suitable for oral, sublingual, subcutaneous, intravenous, epidural, intrathecal, intramuscular, transdermal, pulmonary, local, or rectal administration, and the like, all in unit dosage forms for administration. A preferred route of administration is the oral route.

For oral administration, the active ingredient may be presented as discrete units, such as tablets, capsules, powders, granulates, solutions, suspensions, and the like.

For parenteral administration, the pharmaceutical composition of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

Mixed with such pharmaceutically acceptable auxiliaries, e.g. as described in the standard reference, Gennaro, A. R. et al., Remington: The Science and Practice of Pharmacy (20th Edition, Lippincott Williams & Wilkins, 2000, see especially Part 5: Pharmaceutical Manufacturing), the active agent may be compressed into solid dosage units, such as pills, tablets, or be processed into capsules, suppositories or patches. By means of pharmaceutically acceptable liquids the active agent may be applied as a fluid composition, e.g. as an injection preparation, in the form of a solution, suspension, emulsion, or as a spray, e.g. a nasal spray.

For making solid dosage units, the use of conventional additives such as fillers, colorants, polymeric binders and the like is contemplated. In general any pharmaceutically acceptable additive which does not interfere with the function of the active compounds may be used. Suitable carriers with which the active agent of the invention may be administered as solid compositions include lactose, starch, cellulose derivatives and the like, or mixtures thereof, used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The invention further includes a pharmaceutical composition, as hereinbefore described, in combination with packaging material suitable for said composition, said packaging material including instructions for the use of the composition for the use as hereinbefore described.

The isoxazole-5-carboxamide derivatives of the invention were found to have modulatory properties at the vanilloid receptor (TRPV1 or VR1) as measured by a fluorescence based calcium flux assay using a Chinese Hamster Ovary cell line in which a human recombinant VR1 receptor had been stably expressed. Methods to construct such recombinant cell lines are well known in the art (Sambrook et al., Molecular Cloning: a Laboratory Manual, Cold Spring Harbor Laboratory Press, Cold Spring Harbor, 2000). The compounds of the invention are thus useful in the treatment of TRPV1 mediated disorders, such as in the treatment of acute and chronic pain disorders, acute and chronic neuropathic pain, acute and chronic inflammatory pain, respiratory diseases and in lower urinary tract disorders.

The compounds of the invention may be administered to humans in a sufficient amount and for a sufficient amount of time to alleviate the symptoms. Illustratively, dosage levels for humans may be in the range of 0.001-50 mg per kg body weight, preferably in a dosage of 0.01-20 mg per kg body weight.

The invention is illustrated by the following examples:

### General Methods

Flash column chromatography was performed on silica gel. Semi-preparative high pressure liquid chromatography (semi-prep. HPLC) was performed using the method outlined below:

X-bridge (C 18, 5 μm) 19 mm×50 mm; 10-100% acetonitrile-water over a 8.5 minute gradient followed by 100% acetonitrile for 1.5 minute; 0.1% ammonia buffer; 17 mL/min; detection by UV at 215 nm. Waters Micromass ZQ.

1H NMR coupling constants are given in Hz.

### EXAMPLE 1

### Reference Compound

### N-((1R,3S)-3-Hydroxycyclohexyl)-3-(4-(trifluoromethyl)phenyl)isoxazole-5-carboxamide

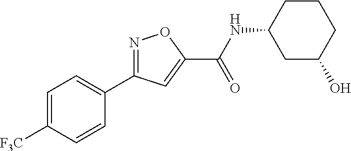

### A: 4-(trifluoromethyl)benzaldehyde oxime

To a mixture of 4-(trifluoromethyl)benzaldehyde (10.0 g, 57.4 mmol) and hydroxylamine hydrochloride (4.47 g, 4.47 mmol) in water (15 mL) and ethanol (15 mL) was added. Ice (30 g) and then potassium hydroxide (15 mL, 150 mmol) was added portionwise. The reaction was stirred at room temperature for 4 hours. The reaction mixture was washed with diethyl ether (20 mL) and then acidified using a 5N HCl solution and the product extracted into dichloromethane (20 mL). The organic layer was washed with brine (20 mL) then dried over sodium sulfate and evaporated to dryness. To the residue was added heptane and the resulting white solid was filtered to afford 4-(trifluoromethyl)benzaldehyde oxime (7.27 g, 38.4 mmol).

### B: N-Hydroxy-4-(trifluoromethyl)benzimidoyl chloride

To a solution of 4-(trifluoromethyl)benzaldehyde oxime (7.27 g, 38.4 mmol) in dimethylformamide (30 mL) was added N-chlorosuccinamide (727 mg, 3.84 mmol). After stirring at room temperature for 30 minutes HCl 2M in diethyl ether (0.1 mL, 0.2 mmol) was added. After a further 30 minutes the remaining N-chlorosuccinimide (6.64 g, 34.5 mmol) was added portionwise over 2 hours. The reaction was stirred at room temperature for 2 hours then allowed to stand overnight. The reaction was poured into ice cold water (150 mL) and extracted with diethyl ether (40 mL), the organic layer was washed with water (2×20 mL) and brine (20 mL). The diethyl ether solution was dried over sodium sulfate and evaporated to dryness to afford N-hydroxy-4-(trifluoromethyl)benzimidoyl chloride (9.1 g, 40.7 mmol).

### C: Ethyl 3-(4-(trifluoromethyl)phenyl)isoxazole-5-carboxylate

A mixture of N-hydroxy-4-(trifluoromethyl)benzimidoyl chloride (2.0 g, 8.95 mmol), ethyl propiolate (0.91 ml, 8.95 mmol) and triethylamine (1.26 ml, 8.95 mmol) in toluene (50 mL) was heated at 60° C. overnight. The reaction was washed with water (3×20 mL) then brine (20 mL) and then dried over sodium sulphate before being filtered and the filtrate evaporated to dryness. Purification by silica gel column chromatography eluting with 10% ethylacetate in heptane afforded ethyl 3-(4-(trifluoromethyl)phenyl)-isoxazole-5-carboxylate (1.5 g, 5.26 mmol).

### D: 3-(4-(Trifluoromethyl)phenyl)isoxazole-5-carboxylic acid

To a solution of ethyl 3-(4-(trifluoromethyl)phenyl)isoxazole-5-carboxylate (305 mg, 1.07 mmol) in tetrahydrofuran (2 mL) and water (1 mL), an aqueous solution of 1N LiOH (1.60 μL, 1.60 mmol) was added. The mixture was stirred at room temperature for 2.5 hours, the reaction mixture was acidified using a 1N HCl solution and the solvent was removed in vacuo obtain 3-(4-(trifluoromethyl)phenyl)isoxazole-5-carboxylic acid (270 mg, 1.05 mmol).

### E: 3-4-(Trifluoromethyl)phenyl)-N-(1R,3S)-3-hydroxycyclohexyl)isoxazole-5-carboxamide

Propanephosphonic acid cyclic anhydride, 50 wt % solution in ethyl acetate (125 μL, 0.21 mmol) was added to a mixture of 3-(4-(trifluoromethyl)phenyl)isoxazole-5-carboxylic acid (36 mg, 0.14 mmol), (1S,3R)-3-aminocyclohexanol (16.1 mg, 0.14 mmol) and diisoproylethylamine (69 μL, 0.42 mmol) in dichloromethane (5.0 mL). After stirring for 1.5 h, the reaction was washed with sodium bicabonate solution and evaporated to dryness in vacuo. The compound was purified by silica gel chromatography eluting with ethyl acetate to afford the title compound: (10 mg, 0.28 mmol). MS (ESI) m/z (M+H⁺): 355.0

The method of Example 1 was further used to prepare the following compounds using alternative amines instead of (1S,3R)-3-aminocyclohexanol.

### EXAMPLE 2(B)

### 4-Chloro-N-cyclopentyl-3-(4-(trifluoromethyl)phenyl)isoxazole-5-carboxamide

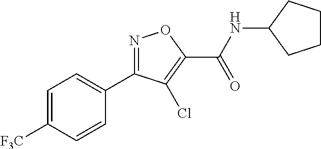

### A: 4-Chloro-3-(4-(trifluoromethyl)phenyl)isoxazole-5-carboxylic acid

The title compound was prepared according to Example 3; Steps B-E.

### B: 4-Chloro-N-cyclopentyl-3-(4-(trifluoromethyl)phenyl)isoxazole-5-carboxamide

The title compound was prepared according to Example 2; Step E using 4-chloro-3-(4-(trifluoromethyl)phenyl)isoxazole-5-carboxylic acid, in place of 3-(4-(trifluoromethyl)phenyl)isoxazole-5-carboxylic acid.

MS (ESI) m/z (M+H⁺): 361.0

### EXAMPLE 2(C)

### 4-Chloro-N-(tetrahydro-2H-pyran-4-yl)-3-(4-(trifluoromethyl)phenyl)isoxazole-5-carboxamide

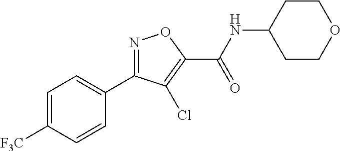

The title compound was prepared according to Example 2(B).

¹H-NMR (400 MHz, CD₃OD) δ 8.08 (d, 2H), 7.88 (d, 2H), 4.14 (m, 1H), 4.00 (d, 2H), 3.52 (t, 2H), 1.90 (d, 2H), 1.71 (m, 2H).

### EXAMPLE 2(D)

### 4-Chloro-N-cyclopentyl-N-methyl-3-(4-(trifluoromethyl)phenyl)isoxazole-5-carboxamide

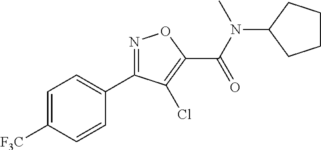

The title compound was prepared according to Example 2(B).

MS (ESI) m/z (M+H⁺): 373.0

### EXAMPLE 2(E)

### (S)-4-Chloro-N-(3-methylbutan-2-yl)-3-(4-(trifluoromethyl)phenyl)isoxazole-5-carboxamide

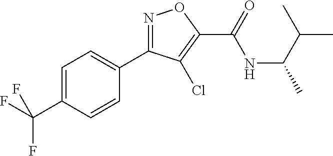

The title compound was prepared according to Example 2(B).

MS (ESI) m/z (M+H⁺): 363.0

### EXAMPLE 2(F) (R)-4-Chloro-N-(1-hydroxybutan-2-yl)-3-(4-(trifluoromethyl)phenyl)isoxazole-5-carboxamide

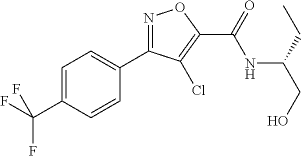

The title compound was prepared according to Example 2(B).

MS (ESI) m/z (M+H⁺): 364.0

### EXAMPLE 2(G)

### (S)-4-Chloro-3-(4-(trifluoromethyl)phenyl)-N-(1,1,1-trifluoropropan-2-yl)isoxazole-5-carboxamide

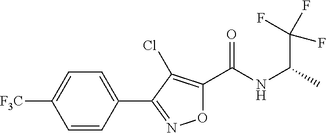

The title compound was prepared according to Example 2(B).

MS (ESI) m/z (M+H⁺): 388.0

### EXAMPLE 2(H)

### 4-Chloro-N-(3,3-difluorocyclobutyl)-3-(4-(trifluoromethyl)phenyl)isoxazole-5-carboxamide

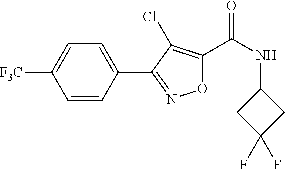

The title compound was prepared according to Example 2(B).

MS (ESI) m/z (M−H)⁻: 379.0

### EXAMPLE 3

### 4-Chloro-N-((1R,3S)-3-hydroxycyclohexyl)-3-(4-(trifluoromethyl)phenyl)isoxazole-5-carboxamide

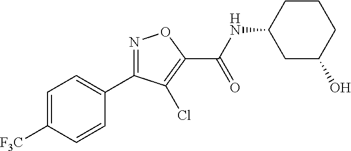

### A: N-Hydroxy-4-(trifluoromethyl)benzimidoyl chloride

The title compound was synthesised according to Example 1; Steps A-B.

### B: (3-(4-(Trifluoromethyl)phenyl)isoxazol-5-yl)methanol

To a mixture of N-hydroxy-4-(trifluoromethyl)benzimidoyl chloride (5.57 g, 24.9 mmol) and prop-2-yn-1-ol (1.45 mL, 24.9 mmol) in toluene (200 mL) was added triethylamine (3.85 mL, 27.4 mmol). After stirring at room temperature for 2 h the reaction was allowed to stand overnight at room temperature. The reaction was washed with water (2×30 mL), followed by brine, the toluene solution was dried over sodium sulfate and evaporated to dryness. Diethyl ether was added to the residue followed by heptane to afford (3-(4-(trifluoromethyl)phenyl)isoxazol-5-yl)methanol (4.38 g, 18.0 mmol) collected.

### C: (4-4-Chloro-3-(4-(trifluoromethyl)phenyl)isoxazol-5-yl)methyl acetate

A mixture of (3-(4-(trifluoromethyl)phenyl)isoxazol-5-yl)methanol (1.0 g, 4.11 mmol), N-chlorosuccinamide (0.66 g, 4.93 mmol) and concentrated sulphuric acid (0.5 mL) in glacial acetic acid (20 mL) was heated at 120° C. for 5 hours. The reaction was poured into water (100 mL) and extracted into ethyl acetate (20 mL) then washed with water (2×20 mL), neutralised aqueous sodium carbonate (5%^{w}/v) then brine. The organic layer was dried with anhydrous sodium sulfate and evaporated to dryness to afford (4-chloro-3-(4-(trifluoromethyl)phenyl)isoxazol-5-yl)methyl acetate (1.18 g, 3.69 mmol).

### D: (4-4-Chloro-3-(4-(trifluoromethyl)phenyl)isoxazol-5-yl)methanol

A solution of (4-chloro-3-(4-(trifluoromethyl)phenyl)isoxazol-5-yl)methyl acetate (1.05 g, 3.28 mmol) and lithium hydroxide in methanol (1M, 4.93 mL, 4.93 mmol) in tetrahydrofuran (10 mL) and water (10 mL) was heated at 60° C. for 6 hours. The reaction was neutralised with hydrochloric acid (2N). The tetrahydrofuran was distilled off and the aqueous residue extracted with ethyl acetate, the organic layer was dried over sodium sulfate and evaporated to dryness to afford (4-chloro-3-(4-(trifluoromethyl)phenyl) isoxazol-5-yl)methanol (0.85 g, 3.09 mmol).

### E: 4-Chloro-3-(4-(trifluoromethyl)phenyl)isoxazole-5-carboxylic acid

A solution of (4-chloro-3-(4-(trifluoromethyl)phenyl) isoxazol-5-yl)methanol (3.7 g, 13.3 mmol), sodium dihydrogenphosphate (0.21 g, 1.73 mmol) and 2,2,6,6-tetra-methylpiperidine oxide (146 mg, 0.93 mmol) in acetonitrile (60 mL) was heated to 35° C. and a solution of sodium chlorite (3.01 g, 26.7 mmol) in water (12 mL) simultaneously as bleach (0.36 mL, 0.27 mmol) in water (6 mL) from different dropping funnels, the reaction becomes very dark. The reaction was heated at 35° C. for 4.5 hours. On cooling sodium sulfite (4.03 g, 32.0 mmol) was added and the mixture stirred for 30 minutes. More water was added to the reaction, this was washed with ethyl acetate, the aqueous phase was then acidified with dilute hydrochloric acid. The product was extracted into ethyl acetate and washed with brine then dried over sodium sulfate and evaporated to dryness to afford 4-chloro-3-(4-(trifluoromethyl)phenyl)isoxazole-5-carboxylic acid (1.8 g, 6.17 mmol).

### F: 4-Chloro-N-((1R,3S)-3-hydroxycyclohexyl)-3-(4-(trifluoromethyl)phenyl)isoxazole-5-carboxamide

A solution of hydroxybenzotriazole (26.3 mg, 0.17 mmol), EDCI (32.9 mg, 0.17 mmol) and 4-chloro-3-(4-(trifluoromethyl)phenyl)isoxazole-5-carboxylic acid were stirred at room temperature for 20 minutes before addition of (1S,3R)-3-aminocyclo-hexanol (21.7 mg, 0.19 mmol) followed by triethylamine (50 μL) stirring was continue for 2 hours. Water was added to the solution then the organic layer separated off and evaporated to dryness. The crude mixture was purified by silica gel column chromatography eluting with 50% ethylacetate in heptane followed by semi prep HPLC to afford the title compound: (37 mg, 0.095 mmol). MS (ESI) m/z (M+H⁺): 389.0

### EXAMPLE 4

### 4-Bromo-N-cyclopentyl-3-(4-(trifluoromethyl)phenyl)isoxazole-5-carboxamide

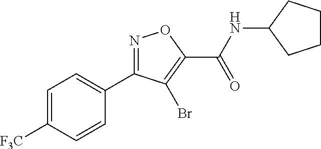

### A: (4-Bromo-3-(4-(trifluoromethyl)phenyl)isoxazol-5-yl)methyl acetate

A mixture of (3-(4-(trifluoromethyl)phenyl)isoxazol-5-yl)methanol (1.0 g, 4.11 mmol), N-bromosuccinamide (0.89 g, 4.93 mmol) and concentrated sulphuric acid (0.5 mL) in glacial acetic acid (20 mL) was heated at 120° C. for 5 hours. The reaction was poured into water (100 mL) and extracted into ethyl acetate (20 mL) then washed with water (2×20 mL), neutralised aqueous sodium carbonate (5%^{w}/v) then brine. The organic layer was dried over anhydrous sodium sulfate and evaporated to dryness to afford (4-bromo-3-(4-(trifluoromethyl)phenyl)isoxazol-5-yl)methyl acetate (1.4 g, 3.84 mmol).

### B: 4-Bromo-3-(4-(trifluoromethyl)phenyl)isoxazole-5-carboxylic acid

The title compound was prepared according to Example 3; Steps D-E whereby in Step D (4-chloro-3-(4-(trifluoromethyl)phenyl)isoxazol-5-yl)methyl acetate was replaced by (4-bromo-3-(4-(trifluoromethyl)phenyl)isoxazol-5-yl)methyl acetate.

### C: 4-Bromo-N-cyclopentyl-3-(4-(trifluoromethyl)phenyl)isoxazole-5-carboxamide

The title compound was prepared according to Example 1; using 4-bromo-3-(4-(trifluoromethyl)phenyl)isoxazole-5-carboxylic acid, in place of 3-(4-(trifluoromethyl)-phenyl)isoxazole-5-carboxylic acid. MS (ESI) m/z (M+H⁺): 405.0

The method of Example 4 was further used to prepare the following compound using alternative amines instead of cyclopentylamine.

### EXAMPLE 5

### 4-Bromo-N-(tetrahydro-2H-pyran-4-yl)-3-(4-(trifluoromethyl)phenyl)isoxazole-5-carboxamide

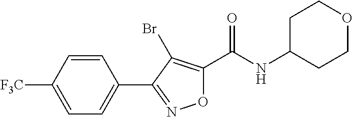

MS (ESI) m/z (M+H⁺): 419.0, 421.0

### EXAMPLE 6

### (S)-4-Bromo-3-(4-(trifluoromethyl)phenyl)-N-(1,1,1-trifluoropropan-2-yl)isoxazole-5-carboxamide

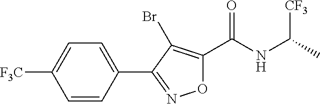

MS (ESI) m/z (M−H)⁻: 431.0

### EXAMPLE 7

### 4-Chloro-3-(4-fluorophenyl)-N-((1R,3S)-3-hydroxycyclohexyl)isoxazole-5-carboxamide

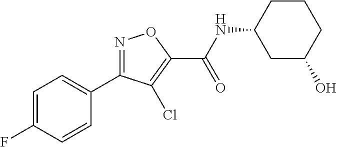

### A: N-Hydroxy-(4-fluoro)benzimidoyl chloride

The title compound was synthesised according to Example 1; Steps A-B whereby in Step A, 4-fluorobenzaldehyde was used in place of 4-(trifluoromethyl)benzaldehyde.

### B: 4-Chloro-3-(4-fluorophenyl)isoxazole-5-carboxylic acid

The title compound was synthesised according to Example 3; Steps B-E whereby in Step B, N-hydroxy-(4-fluoro)benzimidoyl chloride was used in place of N-hydroxy-4-(trifluoromethyl)benzimidoyl chloride.

### C: 4-Chloro-3-(4-fluorophenyl)-N-((1R,3S)-3-hydroxycyclohexyl)isoxazole-5-carboxamide

The title compound was prepared as according to Example 3; Step F whereby 4-chloro-3-(4-fluorophenyl)isoxazole-5-carboxylic acid was used in place of 4-chloro-3-(4-(trifluoromethyl)phenyl)isoxazole-5-carboxylic acid.

MS (ESI) m/z (M+H⁺): 339.0

### EXAMPLE 8

### (S)-4-Chloro-3-(4-fluorophenyl)-N-(1,1,1-trifluoropropan-2-yl)isoxazole-5-carboxamide

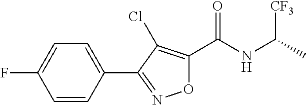

The title compound was prepared as according to Example 7.

MS (ESI) m/z (M−H)⁻: 335.0

### EXAMPLE 9

### 4-Chloro-3-(4-chloro-3-fluorophenyl)-N-(cis)-2-hydroxycyclohexyl)isoxazole-5-carboxamide

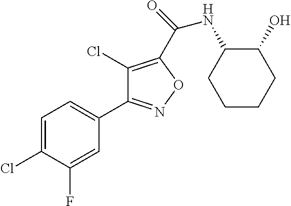

### A: N-Hydroxy-(4-chloro-3-fluoro)benzimidoyl chloride

The title compound was synthesised according to Example 1; Steps A-B whereby in Step A, 4-chloro-3-fluorobenzaldehyde was used in place of 4-(trifluoromethyl)benzaldehyde.

### B: 4-Chloro-3-(4-chloro-3-fluorophenyl)isoxazole-5-carboxylic acid

The title compound was synthesised according to Example 3; Steps B-E whereby in Step B, N-hydroxy-(4-chloro-3-fluoro)benzimidoyl chloride was used in place of N-hydroxy-4-(trifluoromethyl)benzimidoyl chloride.

### C: 4-Chloro-3-(4-chloro-3-fluorophenyl)-N-((1R,3S)-3-hydroxycyclohexyl)isoxazole-5-carboxamide

The title compound was prepared as according to Example 3; Step F whereby 4-chloro-3-(4-chloro-3-fluorophenyl)isoxazole-5-carboxylic acid was used in place of 4-chloro-3-(4-(trifluoromethyl)phenyl)isoxazole-5-carboxylic acid.

MS (ESI) m/z (M+H⁺): 373.0

The method of Example 9 was further used to prepare the following compound using alternative amines instead of cis-2-aminocyclohexanol.

### EXAMPLE 10

### 4-Chloro-3-(4-chloro-3-fluorophenyl)-N-((1R,3S)-3-hydroxycyclohexyl)isoxazole-5-carboxamide

¹H-NMR (400 MHz, CD₃OD) δ 7.78 (d, 1H), 7.70 (m, 2H), 3.97 (m, 1H), 3.69 (m, 1H), 2.16 (d, 1H), 1.93-1.85 (m, 3H), 1.46-1.29 (m, 3H), 1.29-1.21 (t, 1H).

### EXAMPLE 11

### 4-Chloro-3-(3-fluoro-4-(trifluoromethyl)phenyl)-N-(trans)-(2-hydroxycyclohexyl) isoxazole-5-carboxamide

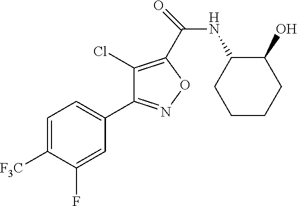

### A: N-Hydroxy-(3-fluoro-4-(trifluoromethyl))benzimidoyl chloride

The title compound was synthesised according to Example 1; Steps A-B whereby in Step A, 3-fluoro-4-(trifluoromethyl)benzaldehyde was used in place of 4-(trifluoromethyl)benzaldehyde.

### B: 4-Chloro-3-(3-fluoro-4-(trifluoromethyl)phenyl)isoxazole-5-carboxylic acid

The title compound was synthesised according to Example 3; Steps B-E whereby in Step B, N-hydroxy-(3-fluoro-4-(trifluoromethyl))benzimidoyl chloride was used in place of N-hydroxy-4-(trifluoromethyl)benzimidoyl chloride.

### C: 4-Chloro-3-(3-fluoro-4-(trifluoromethyl)phenyl)-N-(trans)-(2-hydroxycyclohexyl) isoxazole-5-carboxamide

The title compound was prepared as according to Example 3; Step F whereby 4-chloro-3-(3-fluoro-4-(trifluoromethyl)phenyl)isoxazole-5-carboxylic acid was used in place of 4-chloro-3-(4-(trifluoromethyl)phenyl)isoxazole-5-carboxylic acid.

MS (ESI) m/z (M+H⁺): 407.1.

### EXAMPLE 12

### 4-Chloro-3-(3-fluoro-4-(trifluoromethyl)phenyl)-N-(tetrahydro-2H-pyran-4-yl)isoxazole-5-carboxamide

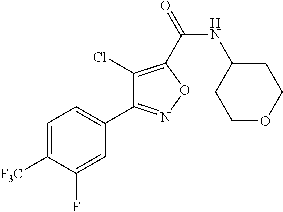

### A: 4-Chloro-3-(3-fluoro-4-(trifluoromethyl)phenyl)isoxazole-5-carboxylic acid

The title compound was synthesised according to Example 11.

### B: 4-Chloro-3-(3-fluoro-4-(trifluoromethyl)phenyl)isoxazole-5-carbonyl chloride

To a suspension of 4-chloro-3-(3-fluoro-4-(trifluoromethyl)phenyl)isoxazole-5-carboxylic acid (340 mg, 1.10 mmol) in dichloromethane (5 mL) was added thionyl chloride (0.12 mL, 1.65 mmol) and the reaction heated under reflux for 6 hour. The solvent was then evaporated off to afford 4-chloro-3-(3-fluoro-4-(trifluoromethyl)phenyl) isoxazole-5-carbonyl chloride (350 mg, 1.07 mmol).

### C: 4-Chloro-3-(3-fluoro-4-(trifluoromethyl)phenyl)-N-(tetrahydro-2H-pyran-4-yl)isoxazole-5-carboxamide

4-Chloro-3-(3-fluoro-4-(trifluoromethyl)phenyl)isoxazole-5-carbonyl chloride (35 mg, 0.11 mmol) was dissolved in anhydrous dichloromethane (1 mL). This was added to a stirred solution of tetrahydro-2H-pyran-4-amine (11.9 mg, 0.12 mmol) and diisopropyl-ethylamine (26.5 mL, 0.16 mmol) in dichloromethane and the reaction was stirred at room temperature for 2 hours. The reaction was washed with water then evaporated to dryness. Purification by silica gel column chromatography eluting with 50% ethylacetate in heptane afforded the title compound: (30 mg, 0.08 mmol)

MS (ESI) m/z (M+H⁺): 393.0

### EXAMPLE 13

### N-Cyclopentyl-3-(3-fluoro-4-(trifluoromethyl)phenyl)-4-(hydroxymethyl)isoxazole-5-carboxamide

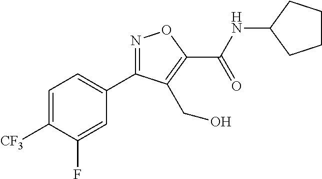

### A: N-Hydroxy-(3-fluoro-4-(trifluoromethyl))benzimidoyl chloride

The title compound was synthesised according to Example 1; Steps A-B whereby in Step A, 3-fluoro-4-(trifluoromethyl)benzaldehyde was used in place of 4-(trifluoromethyl)benzaldehyde.

### B: Dimethyl 3-(3-fluoro-4-(trifluoromethyl)phenyl)isoxazole-4,5-dicarboxylate

To a mixture of 3-fluoro-N-hydroxy-4-(trifluoromethyl)benzimidoyl chloride (1.0 g, 4.14 mmol) and dimethyl but-2-ynedioate (0.59 g, 4.14 mmol) in toluene (15 mL), triethylamine (0.64 mL, 4.55 mmol) was added. The reaction mixture was heated in a microwave at 100° C. for 20 minutes. The reaction mixture was washed with water (2×10 mL), the organic layer dried with anhydrous sodium sulphate, filtered and evaporated to dryness. The crude mixture was purified by silica gel column chromatography eluting with 50% ethylacetate in heptane to give dimethyl 3-(3-fluoro-4-(trifluoromethyl)phenyl)isoxazole-4,5-dicarboxylate (0.65 g, 1.87 mmol)

### C: Methyl 5-(cyclopentylcarbamoyl)-3-(3-fluoro-4-(trifluoromethyl)phenyl)isoxazole-4-carboxylate.

To a solution of dimethyl 3-(3-fluoro-4-(trifluoromethyl)phenyl)isoxazole-4,5-dicarboxylate (1.0 g, 2.88 mmol) in methanol (30 mL) was added cyclopentanamine (0.49 g, 5.76 mmol) dropwise. Stirred for 30 minutes then left at room temperature overnight. The solvent was evaporated off and the residue was partitioned between water and ethylacetate (50 mL), the organic layer was washed with dil HCl (30 mL), water (30 mL) and brine (30 mL) before evaporating to dryness. The residue was triturated in ether (10 mL) and the resulting product filtered to afford methyl 5-(cyclopentylcarbamoyl)-3-(3-fluoro-4-(trifluoromethyl)phenyl)isoxazole-4-carboxylate, (0.68 g, 1.7 mmol).

### D: N-Cyclopentyl-3-(3-fluoro-4-(trifluoromethyl)phenyl)-4-(hydroxymethyl)isoxazole-5-carboxamide

To a stirred solution of methyl 5-(cyclopentylcarbamoyl)-3-(3-fluoro-4-(trifluoromethyl)phenyl)isoxazole-4-carboxylate (0.66 g, 1.65 mmol) in methanol (20 mL) sodium borohydride (125 mg, 3.30 mmol) was slowly added. The reaction was stirred at room temperature and after 2 hours more sodium borohydride (40 mg, 1.05 mmol) was added. After another 2 hours the solvent was evaporated off, the residue was partitioned between ethyl acetate (50 mL) and water (30 mL) then acidified with dil HCl and the organic layer was washed water (2×30 mL) and brine (30 mL) before evaporation to dryness. The residue was purified by silica gel column chromatography eluting with 50% ethylacetate in heptane to afford the title compound: (70 mg, 0.19 mmol). MS (ESI) m/z (M+H⁺): 373.0

### EXAMPLE 14

### N-Cyclopentyl-4-((ethyl(isopropyl)amino)methyl)-3-(3-fluoro-4-(trifluoromethyl) phenyl)isoxazole-5-carboxamide

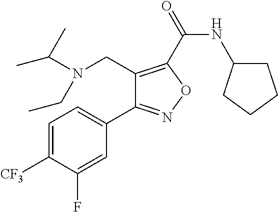

### A: 4-(Chloromethyl)-N-cyclopentyl-3-(3-fluoro-4-(trifluoromethyl)phenyl)isoxazole-5-carboxamide.

To solution of N-cyclopentyl-3-(3-fluoro-4-(trifluoromethyl)phenyl)-4-(hydroxylmethyl)isoxazole-5-carboxamide (90 mg, 0.24 mmol) in N-methyl-2-pyrrolidinone (2 mL), diisopropylethylamine (80 μl; 0.48 mmol) was added followed by methanesulfonyl chloride (28 μL, 0.36 mmol). The reaction was irradiated with microwaves for 1 hour at 180° C., before the addition of methanesulfonyl chloride (28 μL, 0.36 mmol) and diisopropylethylamine (80 μl; 0.48 mmol). The reaction was irradiated with microwaves for a further 1 hour at 180° C. before being poured into water (20 mL) and extracted into ethyl acetate (5 mL), the organic layer was washed with water (2×10 mL) then evaporated to dryness. The product was purified by silica gel column chromatography eluting with 50% ethylacetate in heptane to afford 4-(chloromethyl)-N-cyclopentyl-3-(3-fluoro-4-(trifluoromethyl)phenyl)isoxazole-5-carboxamide (30 mg, 0.08 mmol).

### B: N-Cyclopentyl-4-((ethyl(isopropyl)amino)methyl)-3-(3-fluoro-4-(trifluoromethyl) phenyl)isoxazole-5-carboxamide

A solution of 4-(chloromethyl)-N-cyclopentyl-3-(3-fluoro-4-(trifluoromethyl)phenyl)isoxazole-5-carboxamide (30 mg, 0.08 mmol), N-ethylpropan-2-amine (93 μL, 0.77 mmol) and diisopropylethylamine (51 μL, 0.31 mmol) in acetonitrile (1 mL) was irradiated with microwaves at 180° C. for 30 minutes. The crude mixture was evaporated to dryness and purified by silica gel column chromatography eluting with 5% methanol in dichloromethane to afford the title compound: (24 mg, 0.05 mmol).

MS (ESI) m/z (M+H⁺): 442.2

The method of Example 14 was further used to prepare the following compound using alternative amines instead of N-ethylpropan-2-amine.

### EXAMPLE 15

### 3-(3-Fluoro-4-(trifluoromethyl)phenyl)-4-(morpholinomethyl)-N-(tetrahydro-2H-pyran-4-yl)isoxazole-5-carboxamide

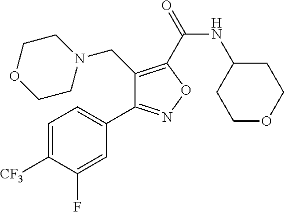

MS (ESI) m/z (M+H⁺): 458.2

### EXAMPLE 16

### Vanilloid Receptor Binding Assay

Test compounds were prepared as stock solution in dimethylsulfoxide and tested for activity over several log units (ranging 100 μM-100 μM). Compounds were further diluted in assay buffer as necessary for IC₅₀ determination.

Chinese hamster ovary cells expressing human VR1 were grown in DMEM/F12 50/50 Mix (Mediatech, Inc., Herndon, Va., USA), supplemented with 10% FetalClone II (Hyclone, Logan, Utah, USA), 1% GlutaMax (Invitrogen Corp., Carlsbad, Calif., USA), 1% Pen/Strep (Mediatech) and 0.4 mg/ml G418 (Mediatech). The day before the assay, cells were seeded into 384-well tissue culture-treated black plates with clear bottoms (Corning, Inc., Corning, N.Y., USA), at 10,000 viable cells/well in 50 μl/well of medium containing no G418.

On the day of the assay, which is the FLIPR® Calcium 3 Assay commercially available from Molecular Devices Corp., Sunnyvale, Calif. USA, the plating medium was removed and replaced with 25 μl/well 1× Calcium 3 Assay kit dye, prepared in VR1 Buffer (160 mM NaCl, 4.5 mM KCl, 10 mM HEPES, 10 mM Glucose, 2 mM CaCl₂, 1 mM MgCl₂ and 0.5 mM Probenecid). After 1 hour incubation at room temperature, the plates were loaded into the FLIPR (Molecular Devices, Corp.), which adds 12.5 μA of test compound in VR1 Buffer containing 4% dimethylsulfoxide and reads the subsequent change in the fluorescence of the cells to monitor agonist activity. Ten minutes after compound addition, the plates were reloaded into the FLIPR, which adds 12.5 μA of 30 nM capsaicin in VR1 Buffer and reads the subsequent change in the fluorescence of the cells to monitor antagonist activity. In this way, the same assay was used to assess both the agonist activity and antagonist activity of test compounds.

Typical IC₅₀ values measured in the in vitro assay described above for the compounds of the invention are 10 μM or less. For several embodiments of the invention the IC₅₀ was found to be below 100 nM.

1-8. (canceled)

9. An isoxazole-5-carboxamide derivatives having the general Formula I 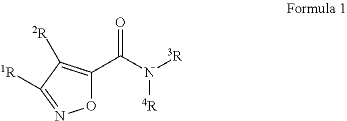
wherein
R₁ is phenyl or pyridyl, each of which optionally substituted by 1-3 substituents selected from halogen, (C₁₋₄)alkyl, halo(C₁₋₄)alkyl, (C₁₋₄)alkyloxy and halo(C₁₋₄)alkyloxy;
R₂ is halogen, (C₁₋₃)alkyl, hydroxy(C₁₋₃)alkyl, (C₁₋₄)alkyloxy(C₁₋₃)alkyl, (C₃₋₈)cycloalkyl, hydroxy(C₃₋₈)cycloalkyl or R₅R₆N(C₁₋₃)alkyl;
R₃ is (C₁₋₈)alkyl, halo(C₁₋₈)alkyl, hydroxy(C₁₋₈)alkyl, (C₂₋₈)alkenyl, (C₂₋₈)alkynyl, (C₃₋₁₀)cycloalkyl, (C₃₋₈)cycloalkenyl or (C₃₋₈)cycloalkyl(C₁₋₃)alkyl, each cycloalkyl group optionally substituted by oxo, hydroxyimino, hydroxy, carboxy, cyano, (C₁₋₃)alkyl and hydroxy(C₁₋₃)alkyl; or
R₃ is a saturated 4-8-membered heterocyclic ring containing a heteroatom selected from O, S and SO₂, optionally substituted by hydroxyl or oxo;
R₄ is H or (C₁₋₄)alkyl; or
R₄ together with R₃ and the N to which they are bonded form a saturated 4-8 membered ring, optionally containing a further heteroatom selected from O, S and SO₂, the ring being optionally substituted by oxo, hydroxyimino, hydroxy, carboxy, carboxamido, (C₁₋₃) alkyl, hydroxy(C₁₋₃)alkyl or (C₁₋₃)-alkyloxy;
R₅ and R₆ are independently H, (C₁₋₆)alkyl, (C₃₋₆)cycloalkyl or (C₃₋₆)cycloalkyl-(C₁₋₃)alkyl, each alkyl group being optionally substituted with halogen, hydroxy or (C₁₋₄)alkyloxy; or
R₅ and R₆ form together with the nitrogen to which they are bonded a 5- or 6-membered saturated heterocyclic ring, optionally comprising a further heteroatom selected from O, S and SO₂; or a pharmaceutically acceptable salt thereof.

10. The isoxazole-5-carboxamide derivative of claim 9, wherein
R₁ is phenyl, optionally substituted by 1-3 substituents selected from halogen, (C₁₋₄)alkyl, halo(C₁₋₄alkyl, (C₁₋₄)alkyloxy and halo(C₁₋₄)alkyloxy;
R₂ is halogen, hydroxy(C₁₋₃)alkyl or R₅R₆N(C₁₋₃)alkyl;
R₃ is (C₁₋₈)alkyl, halo(C₁₋₈)alkyl, hydroxy(C₁₋₈)alkyl or (C₃₋₁₀)cycloalkyl, optionally substituted by hydroxy; or
R₃ is a saturated 4-8-membered heterocyclic ring containing a heteroatom selected from O, S and SO₂, optionally substituted by hydroxyl or oxo;
R₄ is H or (C₁₋₄)alkyl;
R₅ and R₆ are independently H, (C₁₋₆)alkyl, (C₃₋₆)cycloalkyl or (C₃₋₆)cycloalkyl-(C₁₋₃)alkyl, each alkyl group being optionally substituted with halogen, hydroxy or (C₁₋₄)alkyloxy; or
R₅ and R₆ form together with the nitrogen to which they are bonded a 5- or 6-membered saturated heterocyclic ring, optionally comprising a further heteroatom selected from O, S and SO₂.

11. The isoxazole-5-carboxamide derivative of claim 9, wherein
R₁ is phenyl, substituted by 1 or 2 substituents selected from F, C₁ and CF₃;
R₂ is Cl, Br, hydroxy(C₁₋₃)alkyl or R₅R₆N(C₁₋₃)alkyl;
R₃ is (C₁₋₈)alkyl, halo(C₁₋₈)alkyl, hydroxy(C₁₋₈)alkyl or (C₃₋₁₀)cycloalkyl, optionally substituted by hydroxy; or
R₃ is a saturated 4-8-membered heterocyclic ring containing a heteroatom selected from O, S and SO₂;
R₄ is H or (C₁₋₄)alkyl;
R₅ and R₆ are independently H, (C₁₋₆)alkyl, (C₃₋₆)cycloalkyl or (C₃₋₆)cycloalkyl-(C₁₋₃)alkyl, each alkyl group being optionally substituted with halogen, hydroxy or (C₁₋₄)alkyloxy; or
R₅ and R₆ form together with the nitrogen to which they are bonded a 5- or 6-membered saturated heterocyclic ring, optionally comprising a further heteroatom selected from O, S and SO₂.

12. The isoxazole-5-carboxamide derivative of claim 9 which is selected from
4-chloro-N-((1R,3S)-3-hydroxycyclohexyl)-3-(4-(trifluoromethyl)phenyl)isoxazole-5-carboxamide;
4-bromo-N-cyclopentyl-3-(4-(trifluoromethyl)phenyl)isoxazole-5-carboxamide;
4-bromo-N-(tetrahydro-2H-pyran-4-yl)-3-(4-(trifluoromethyl)phenyl)isoxazole-5-carboxamide;
4-chloro-N-cyclopentyl-3-(4-(trifluoromethyl)phenyl)isoxazole-5-carboxamide;
4-chloro-3-(4-fluorophenyl)-N-((1R,3S)-3-hydroxycyclohexyl)isoxazole-5-carboxamide;
4-chloro-N-(tetrahydro-2H-pyran-4-yl)-3-(4-(trifluoromethyl)phenyl)isoxazole-5-carboxamide;
4-chloro-N-cyclopentyl-N-methyl-3-(4-(trifluoromethyl)phenyl)isoxazole-5-carboxamide;
(S)-4-chloro-N-(3-methylbutan-2-yl)-3-(4-(trifluoromethyl)phenyl)isoxazole-5-carboxamide;
(R)-4-chloro-N-(1-hydroxybutan-2-yl)-3-(4-(trifluoromethyl)phenyl)isoxazole-5-carboxamide;
(S)-4-bromo-3-(4-(trifluoromethyl)phenyl)-N-(1,1,1-trifluoropropan-2-yl)isoxazole-5-carboxamide;
(S)-4-chloro-3-(4-(trifluoromethyl)phenyl)-N-(1,1,1-trifluoropropan-2-yl)isoxazole-5-carboxamide;
4-chloro-N-(3,3-difluorocyclobutyl)-3-(4-(trifluoromethyl)phenyl)isoxazole-5-carboxamide;
(S)-4-chloro-3-(4-fluorophenyl)-N-(1,1,1-trifluoropropan-2-yl)isoxazole-5-carboxamide;
4-chloro-3-(4-chloro-3-fluorophenyl)-N-(cis)-2-hydroxycyclohexyl)isoxazole-5-carboxamide;
4-chloro-3-(4-chloro-3-fluorophenyl)-N-((1R,3S)-3-hydroxycyclohexyl)isoxazole-5-carboxamide;
N-cyclopentyl-3-(3-fluoro-4-(trifluoromethyl)phenyl)-4-(hydroxymethyl)isoxazole-5-carboxamide;
4-chloro-3-(3-fluoro-4-(trifluoromethyl)phenyl)-N-(trans)-2-hydroxycyclohexyl)isoxazole-5-carboxamide;
4-chloro-3-(3-fluoro-4-(trifluoromethyl)phenyl)-N-(tetrahydro-2H-pyran-4-yl)-isoxazole-5-carboxamide;
N-cyclopentyl-4-((ethyl(isopropyl)amino)methyl)-3-(3-fluoro-4-(trifluoromethyl)phenyl)isoxazole-5-carboxamide; and
3-(3-fluoro-4-(trifluoromethyl)phenyl)-4-(morpholinomethyl)-N-(tetrahydro-2H-pyran-4-yl)isoxazole-5-carboxamide; or a pharmaceutically acceptable salt thereof.

13. A pharmaceutical composition comprising an isoxazole-5-carboxamide derivative of claim 9 or a pharmaceutically acceptable salt thereof and pharmaceutically suitable auxiliaries.

14. A pharmaceutical composition comprising an isoxazole-5-carboxamide derivative of claim 12 or a pharmaceutically acceptable salt thereof and pharmaceutically suitable auxiliaries.

15. A method of treating a human suffering from pain, wherein the pain is selected from the group consisting of acute and chronic pain disorders, acute and chronic neuropathic pain, acute and chronic inflammatory pain, respiratory diseases, and lower urinary tract disorders, the method comprising administering to the human a therapeutically effective amount of an isoxazole-5-carboxamide derivative of claim 9 or a pharmaceutically acceptable salt thereof.

16. A method of treating a human suffering from pain, wherein the pain is selected from the group consisting of acute and chronic pain disorders, acute and chronic neuropathic pain, acute and chronic inflammatory pain, respiratory diseases, and lower urinary tract disorders, the method comprising administering to the human a therapeutically effective amount of an isoxazole-5-carboxamide derivative of claim 12 or a pharmaceutically acceptable salt thereof.
